(19) United States
(12) Patent Application Publication
(10) Pub. No.: US 2011/0091574 A1
(43) Pub. Date: Apr. 21, 2011

(54) TREATMENT OF ADENOCARCINOMA EXPRESSING LKB1 WITH MTOR INHIBITOR IN COMBINATION WITH COX2 INHIBITOR

(75) Inventors: Landon J. Inge, Phoenx, AZ (US); Keith D. Coon, San Diego, CA (US)

(73) Assignee: Catholic Healthcare West, Phoenix, AZ (US)

(21) Appl. No.: 12/865,607

(22) PCT Filed: Feb. 13, 2009
(86) PCT No.: PCT/US09/34106

§ 371 Date: Dec. 14, 2010

Related U.S. Application Data
(60) Provisional application No. 61/029,159, filed on Feb. 15, 2008.

(51) Int. Cl. A61K 33/36; C12N 5/071; A61K 31/415; A61K 31/365; A61K 31/42; A61K 31/444; A61K 31/196; A61K 31/7004; A61K 31/19; A61K 31/4406; A61K 31/51; A61K 31/195; A61K 33/16; A61P 35/00

(52) U.S. Cl. 424/624; 435/325; 514/406; 514/473; 514/378; 514/334; 514/567; 514/23; 514/561; 514/356; 514/276; 424/676

(57) ABSTRACT

Methods of treating adenocarcinoma cells such as NSCLC cells are disclosed that depend upon the level of functionally active LKB1 expressed in the cancer cells being treated. In one embodiment, the cancer cells express functionally active LKB1 and the method comprises contacting those cells with a LKB1-stimulating amount of a COX-2-specific inhibitor in combination with an inhibiting amount of a specific inhibitor of mTOR. In another embodiment, the cancer cells express about 25 percent or less of the normal, functional LKB1 expressed by non-transformed cells of the same type are contacted with a growth inhibiting amount of an agent that inhibits cellular metabolism and induces energetic stress.

### TECHNICAL FIELD

The present invention relates to the relation of enzyme designated LKB1 and adenocarcinoma treatments. More particularly, the invention relates to the use of the presence or absence of the LKB1 enzyme in cancer cells as an indication of an appropriate drug treatment for an adenocarcinoma such as non-small cell lung cancer (NSCLC).

### BACKGROUND ART

Although recent statistics reveal significant increases in survival for many cancers, survival rates for lung cancer have seen little improvement over the last few decades, with overall patient 5-year survival approximately 15% [Jemal et al., CA Cancer J Clin 2007 (Jan. 1, 2007) 57(1):43-66]. Non-small cell lung cancer (NSCLC) accounts for the majority (about 80%) of lung cancers compared to the other subtypes [Travis et al., Cancer 1995 75(1 Suppl):191-202].

Currently, curative treatment of non-small cell lung cancer (NSCLC) is restricted to surgical resection for very early disease, with the addition of systemic chemotherapy and radiation treatment for patients with later stages of cancer, since metastatic recurrence is common. Unfortunately, the response rates for NSCLC to traditional chemotherapeutic agents has been poor, although the more recent attempts at personalizing chemotherapy appear to hold promise for improving outcomes. Certainly, the understanding of epidermal growth factor receptor (EGFR) gene mutations in some cancers has helped direct application of tyrosine kinase inhibitors towards patients with these distinct mutations [Sharma et al., Nature Reviews 2007 (March) 7(3):169-181]. The success of this approach has stimulated the idea that certain characteristics of each individual cancer can be exploited to improve outcomes for the disease. Thus, the paradigm in lung cancer is changing toward one of personalized therapy.

The LKB1 tumor suppressor gene is commonly mutated in NSCLC, and offers a therapeutic opportunity. The LKB1 gene was discovered through genetic linkage analysis of the familial disorder, Peutz-Jeghers syndrome (PJS) [Alessi et al., Annual Review of Biochemistry 2006 75:137-163], and has since been found to be inactivated in 30%-50% of NSCLC patients [Matsumoto et al. Oncogene 2007 (Aug. 30) 26(40):5911-5918; Sanchez-Cespedes et al. Cancer Research 2002 (Jul. 1) 62(13):3659-3662; and Ramsey et al. Nature 2007 (Aug. 16) 448(7155):807-810], about twice the prevalence of EGFR mutations found in this disease. Further, convergent in vitro and in vivo studies have led to the realization that loss of LKB1 may be a critical event in NSCLC [Ramsey et al. Nature 2007 (Aug. 16) 448(7155):807-810; Sanchez-Cespedes Oncogene 2007 (Jun. 18) 7825-32; Shah et al., Cancer Research 2008 (May 15) 68(10):3562-3565; and Shaw, Current Opinion in Cell Biology 2006 December 18(6):598-608].

LKB1 (also known as STK11 serine/threonine kinase 11) is a serine-threonine kinase, phosphorylating and regulating 14 different protein kinases [Alessi et al., Annual Review of Biochemistry 2006 75:137-163]. The biological role of LKB1 regulation of these kinases remains largely unknown except for the AMP-activated kinase, or AMPK. The primary function of LKB1-AMPK signaling is in the regulation of cellular energy metabolism. Increases in intracellular levels of AMP, due to hypoxia, ischemia, or other stressors, induce the LKB1 dependent activation of AMPK, allowing AMPK to alter cellular functions and restore ATP levels within the cell [Alessi et al., Annual Review of Biochemistry 2006 75:137-163; and Shaw, Current Opinion in Cell Biology 2006 December 18(6):598-608].

Cell growth is a process regulated by AMPK during low energy states [Inoki et al., Cell 2003 (Nov. 26) 115(5):577-590]. As deregulation of cell growth is a critical feature of cancer, this function of LKB1 and AMPK has led to an increased interest in their roles in malignancy.

During the 1920's, Otto Warburg made the discovery that tumor cells rely upon glycolysis, as opposed to normal mitochondrial oxidative phosphorylation, for energy production (the ‘Warburg effect’) [Shaw, Current Opinion in Cell Biology 2006 December 18(6):598-608]. Recent work in basic and clinical research has improved this basic understanding, demonstrating that tumor cells have uniquely altered metabolisms, displaying increased glucose uptakes, increases in enzymes of the glycolytic pathway and an increased sensitivity to inhibition of glycolysis [Shaw, Current Opinion in Cell Biology 2006 December 18(6):598-608].

The increased uptake of glucose and lack of glucose-6-phosphatase activity (that directs glucose exit from the cancer cell) has led to the clinical use of the glucose analogue, [¹⁸F] fluoro-deoxyglucose, in positron emission tomography (PET) scanning. Another glucose analog, 2-deoxyglucose (2-DG) inhibits glycolysis, and appears to be preferentially toxic to tumor cell [Karczmar et al., Cancer Research 1992 (Jan. 1) 52(1):71-76; Maschek et al., Cancer Research 2004 (Jan. 1) 64(1):31-34; and Simons et al., Cancer Research 2007 (Apr. 1) 67(7):3364-3370]. Studies have found that 2-DG has potential in treating cancer and is being investigated currently in phase I trials. 2-DG is also being explored as a treatment for epilepsy as a surrogate for the “ketogenic diet” [Garriga-Canut et al., Nat Neurosci 2006 9(11):1382-1387] and it appears to be well tolerated in early studies. Fortuitously, and of significant importance, 2-DG is also a specific activator of LKB1-AMPK signaling, suggesting that LKB1-AMPK may be critical in mediating 2-DG's anti-tumor effects.

LKB1 and AMPK negatively affect cell growth by inhibition of the protein kinase, mTOR (mammalian target of rapamycin), which functions in increasing cell growth and is commonly deregulated in cancer [Guertin et al., Cancer Cell 2007 July 12(1):9-22]. LKB1-AMPK regulation of mTOR occurs via AMPK activation of the TSC1/2 tumor suppressors, which inhibit mTOR activation [Inoki et al., Cell 2003 (Nov. 26) 115(5):577-590; and Corradetti et al., Genes & Development 2004 (Jul. 1) 18(13):1533-1538]. Interestingly, stimuli that normally activate LKB1-AMPK, fail to result in decreased mTOR activity in LKB1 null cells [Corradetti et al., Genes & Development 2004 (Jul. 1) 18(13):1533-1538; Carretero et al., Oncogene 2007 (Mar. 8) 26(11):1616-1625; and Shaw et al., Cancer Cell 2004 July 6(1):91-99] and can activate apoptosis (FIG. 1). These and other findings have established a direct link between the LKB1-AMPK and the mTOR signaling pathways.

Recent studies have found a link between the loss of LKB1 and increased aggressiveness of adenocarcinomas such as NSCLC, colorectal adenoma, prostate and endometrial adenomas. In human endometrial cancers, LKB1 expression was found inversely correlated with tumor grade and stage, implying that LKB1 inactivation or down-regulation also contributes to endometrial cancer progression in women. [Contreras et al., Cancer Research 2008 68(3):759-766.]

Genetic analysis revealed a predilection of increased expression of EGFR and has led to the development of EGFR specific therapies [Sharma et al., Nat Rev Cancer 2007 7(3):169-181]. However, although advances have been made in understanding how the molecular and genetic changes to EGFR contribute to NSCLC, little is known about how genetic and/or molecular changes in other proteins contribute to NSCLC.

For 2007, NSCLC will cause the death of approximately 160,390 people in the United States alone [Jemal et al., CA Cancer J Clin 2007 57(1):43-66]. Although targeted therapies have proven effective for other solid tumors [Herceptin® (trastuzumab), Avastin® (bevacizumab)], the EGFR inhibitor, Tarceva® (erlotinib), is the only FDA approved targeted therapy for the treatment of NSCLC. Currently, Tarceva® is most effective in patients with EGFR mutations, a distinct sub-population of patients, representing only about 10% of NSCLC in the United States [Sharma et al., Nat Rev Cancer 2007 7(3):169-181].

Peutz-Jeghers syndrome (PJS) is characterized by the growth of large benign hamartomas and a 93% risk of developing of malignant tumors. Genetic linkage analysis has demonstrated that mutations of the serine-threonine kinase, LKB1, cause PJS [Alessi et al., Annu Rev Biochem 2006; 75:137-163].

LKB1 has been characterized as a tumor suppressor, yet somatic mutations to LKB1 appear to be rare in most sporadic cancers [Alessi et al., Annu Rev Biochem 2006 75:137-163; Avizienyte et al., Am J Pathol 1999 154(3):677-681]. However, recent work has shown that in lung carcinomas, mutational loss of LKB1 occurs in about 30% to about 50% of cases [Matsumoto et al., Oncogene 2007 26(40):5911-5918; Sanchez-Cespedes et al., Cancer Res 2002 62(13):3659-3662; Memmott et al., Cancer Res. 2008 Jan. 15; 68(2):580-588]. NSCLC is a heterogeneous disease consisting of large cell carcinoma (LCC), adenocarcinoma, squamous cell carcinoma (SCC) and mixed histology tumors (adenosquamous). Among these subtypes, LKB1 loss appears to occur most frequently in adenocarcinoma (34%), with LKB1 loss occurring in SCC (19%), LCC (14%) and adenosquamous (25%) at lower rates [Shah et al., Cancer Research 2008 (May 15) 68(10):3562-3565].

Further, LKB1 loss synergistically cooperates with oncogenic gene KRAS to decrease tumor latency and increase tumor metastasis in a transgenic mouse model of lung cancer [Ji et al., Nature 2007 448(7155):807-810]. The KRAS gene provides instructions for making a protein (called K-Ras) that is involved primarily in regulating cell division. The protein relays signals from outside the cell to the cell nucleus. These signals instruct the cell to grow and divide or to mature and take on specialized functions (differentiate).

The K-Ras protein is a GTPase that converts GTP into GDP. The K-Ras protein acts like a switch, and it is turned on and off by the GTP and GDP molecules. To transmit signals, the K-Ras protein must be turned on by binding to a molecule of GTP. The K-Ras protein is turned off (inactivated) when it converts the GTP to GDP. When the protein is bound to GDP, it does not relay signals to the cell nucleus.

Mechanistically, LKB1 functions at the center of a complex signaling network, phosphorylating and activating 14 protein kinases [Alessi et al., Annu Rev Biochem 2006; 75:137-163]. The best characterized of the LKB1 activated kinases is the AMP-activated kinase, or AMPK. The primary function of LKB1-AMPK signaling is in the regulation of cellular energy metabolism. Increases in intracellular levels of AMP due to hypoxia or ischemia, induce the LKB1 dependent activation of AMPK, allowing AMPK to alter cellular functions and restore ATP levels within the cell [Alessi et al., Annu Rev Biochem 2006; 75:137-163; Shaw, Curr Opin Cell Biol 2006 18(6):598-608].

As noted earlier, one process regulated by AMPK during low energy states is cell growth [Inoki et al., Cell 2003 115(5):577-590]. As deregulation of cell growth is a critical feature of cancer, this function of LKB1 and AMPK has led to an increased interest in their roles in cancer. The protein kinase, mTOR (mammalian target of rapamycin) functions to promote cell growth and is commonly deregulated in cancer [Guertin, Cancer Cell 2007 12(1):9-22]. LKB1 and AMPK negatively regulate mTOR, as AMPK activation of the TSC1/2 tumor suppressors results in inhibition of mTOR [Inoki et al., Cell 2003 115(5):577-590; Corradetti et al., Genes Dev 2004 18(13):1533-1538], whereas stimuli that normally activate LKB1-AMPK, fail to result in decreased mTOR activity in LKB1 null cells [Carretero et al., Oncogene 2007 26(11):1616-1625; Shaw et al., Cancer Cell 2004 6(1):91-99]. These findings as well as additional studies have established a direct link between the LKB1-AMPK and the mTOR signaling pathways.

One potential target for the treatment of NSCLC is the cyclooxygenase-2 (COX-2) enzyme that is over-expressed in NSCLC as well as several other adenocarcinomas [Brown et al., Clin Cancer Res 2004 10(12 Pt 2):4266s-4269s; Dannenberg et al., Cancer Cell 2003 4(6):431-436]. It is the production of a class of bioactive lipids, the prostaglandins, by COX-2 that contributes to the progression of the disease [Brown et al., Clin Cancer Res 2004 10(12 Pt 2):4266s-4269s; Dannenberg et al., Cancer Cell 2003 4(6):431-436]. In addition to activation of signaling pathways [Dannenberg et al., Cancer Cell 2003 4(6):431-436], enzymatic activity of COX-2 also results in inhibition of LKB1 kinase activity [Wagner et al., J Biol Chem 2006 281(5):2598-2604].

Because of the role of COX-2 in cancer, studies have been undertaken to use COX-2 inhibitors as a therapy for cancer. One such drug is celecoxib (Celebrex®), a specific inhibitor to COX-2, which is currently FDA approved in the treatment of pain and inflammation. Another COX-2 inhibitor, rofecoxib (Vioxx®), has been removed from the US market because of the finding of a two-fold increased risk of cardiovascular toxicities in a trial to prevent adenomas. A third COX-2 inhibitor, valdecoxib (Bextra™) was voluntarily withdrawn from the US market. Although celecoxib has shown efficacy in preventing colon carcinoma at high doses (e.g., 400 mg of celecoxib once daily, or 200 mg or 400 mg twice daily), the severe cardiovascular side effects associated with long-term use at these doses have made celecoxib potentially unattractive for use as a preventative or therapeutic drug [Arber et al., N Engl J Med 2006 355(9):885-895; Solomon et al., N Engl J Med 2005 352(11):1071-1080].

Due to the interaction of COX-2 with EGFR, studies have combined celecoxib with EGFR inhibitors resulting in improved efficacy for both drugs in controlling tumor progression [Buchanan et al., Cancer Res 2007 67(19):9380-9388; Reckamp et al., Clin Cancer Res 2006 12(11 Pt 1):3381-3388; Zhang et al., Clin Cancer Res 2005 11(17):6261-6269].

In another function of LKB1, energetic stress activates the enzyme and induces cell cycle arrest as a means to conserve energy. Conversely, cells that lack LKB1 fail to react to such stress and undergo cell death. As noted earlier, somatic loss of LKB1 has been found to occur in about 30% to about 50% of NSCLC, indicating increased cellular susceptibility to therapeutic agents in LKB1 null patients using a therapeutic agent that inhibits cellular metabolism and induces energetic stress, resulting in decreased cellular viability.

In view of the limitations in the use of Tarceva® in the clinic, it is important to develop new therapies to help alleviate the burden of this disease. The invention discussed hereinafter illustrates therapeutic regimens based in part on the presence or absence of functionally active LKB1 in adenocarcinoma cells.

### BRIEF SUMMARY OF THE INVENTION

The findings discussed before indicate that inactivation of LKB1 can be an important event in the progression of non-small cell lung carcinoma (NSCLC). Because of the roles LKB1 and COX-2 can play in NSCLC, one aspect of the invention contemplates inhibition of COX-2 by an appropriate COX-2-specific inhibitor such as celecoxib to restore LKB1 tumor suppressor activity. Further, the addition of a low dose of rapamycin, a specific inhibitor of mTOR, in combination with the COX-2-specific inhibitor provides a potent synergistic effect upon limiting the growth of LKB1 expressing NSCLC tumors. Preliminary studies illustrated herein using low doses of celecoxib and rapamycin support this hypothesis.

Thus, one aspect of the invention contemplates a method of treating adenocarcinoma cells that express LKB1, as do about 70 percent of NSCLC cells. In that method, adenocarcinoma cells such as those of NSCLC that express functionally active LKB1 are contacted with a LKB1-stimulating amount of a COX-2-specific inhibitor such as celecoxib, rofecoxib, valdecoxib, parecoxib, lumiracoxib, etoricoxib and mixtures thereof, in combination with an inhibiting amount of a specific inhibitor of mTOR such as a rapamycin-like macrolide such as rapamycin itself (Rapamune®) or a rapamycin derivative such as temsirolimus [42-(3-hydroxy-2-(hydroxymethyl)-2-methylpropanoate)-rapamycin; also known as CCI-779, Torisel® (temsirolimus)] and everolimus [RAD-001; or Certican®]. The proposed contacting is contemplated to occur multiple times over a period of months or years, such as by daily dosing, at least until the cell number remains constant when carried out in vitro, or the in vivo tumor size stabilizes or declines.

Another aspect of the invention contemplates the absence of functionally active LKB1 in the adenocarcinoma cells being treated, e.g. using LKB1 null cells. These cells can contain about 50 percent or less of the normal, functional LKB1 expressed by non-transformed cells of the same type, such as lung cells in the case of NSCLC. As noted previously, LKB1 is absent from about 30 to about 50 percent of adenocarcinoma cells such as NSCLC cells. Here, a growth inhibiting amount of an agent that inhibits of the glycolytic pathway or cellular metabolism and induces energetic stress, such as 2-deoxyglucose (2-DG), bromopyruvic acid, 6-aminonicotinamide, oxythiamine chloride, sodium arsenate dibasic heptahydrate, sodium oxamate, sodium fluoride and mixtures thereof, is contacted with the LKB1 null adenocarcinoma cells as discussed above.

The invention thus provides a method by which one can individualize treatment of adenocarcinoma cells to enhance the opportunities for killing those cells. In carrying out that individualized method of treatment, one contacts the cells with one or the other of a pharmaceutical composition containing (1) a LKB1-stimulating amount of a COX-2-specific inhibitor in combination with an inhibiting amount of a specific inhibitor of mTOR, or (2) a growth inhibiting amount of an agent that inhibits cellular metabolism and induces energetic stress. In this method, the cells that express functionally active LKB1 are contacted with (1), and cells that express about 25 percent or less of the normal, functional LKB1 expressed by non-transformed cells of the same type are contacted with (2).

The present invention has several benefits and advantages.

One benefit is that its combined use of a COX-2-specific inhibitor and a specific inhibitor of mTOR provides a valuable tool in the treatment of adenocarcinomas that can utilize pharmaceutical products that are or have been approved for use in humans.

An advantage of the invention is that its use of an inhibitor of cellular metabolism and energetic stress inducer for treating cancers that express substantially less than the usual amount of functional LKB1 permits targeted therapy for a relatively larger portion of the population (about 30 to about 50%) as compared to treatment with Tarceva® that is most effective in patients with EGFR mutations, a distinct sub-population of patients, representing only about 10 percent of the affected population.

Another benefit of the invention is that its use can provide a method for determining a personalized therapeutic route to treatment of an adenocarcinoma based on the presence or absence of a functional LKB1 gene.

Still further benefits and advantages will be apparent to the skilled worker from the discussion that follows.

### BRIEF DESCRIPTION OF THE DRAWINGS

In the drawings, forming a portion of this disclosure,

FIG. 1 is a schematic representation of LKB1-AMPK signaling and regulation of mTOR.

FIG. 2 (in two panels, A and B) shows a series of graphs of cell viability percentage versus the concentration of treating agent for H2030 and A549 cells that were treated with celecoxib, rapamycin, or both for 72 hours. Cell viability was determined with the CellTiter™ blue kit and all raw values were normalized to vehicle (DMSO) treatment.

FIG. 3 (in three panels, A, B and C) illustrates the effect of 2-deoxyglucose (2-DG) in LKB1 null and LKB1 expressing NSCLC cells. FIG. 3A is an immunoblot of LKB1 in H23, H2122, H2009 and H441 NSCLC cell lines. GAPDH was used as a loading control. FIG. 3B illustrates an immunoblot showing the induction of AMPK phosphorylation by 2-DG. LKB1 positive (H2009, H441) and LKB1 negative (H23, H2122) cell lines were treated with 20 mM of 2-DG for 1 hour. Protein lysates were immunoblotted with an antibody specific to phosphorylated Thr172 AMPK. It is noted that 2-DG induces phosphorylation at Thr172 only in LKB1 expressing cells. Both AMPK and GAPDH were used as loading controls. The blot is representative of two independent experiments. C-LKB1 null NSCLC cell are more sensitive to 2-DG. FIG. 3C is a graph of cell viability versus 2-DG concentration that shows that 2-DG decreases cell viability in LKB1-NSCLC cell lines. NSCLC cell lines were treated for 48 hours with indicated concentrations of 2-DG. Bars represent standard error.

FIG. 4 (in panels A through F) illustrates in graphs and immunoblots that 2-DG induces apoptosis in LKB1 null NSCLC cells. FIG. 4A is a graph that shows the activity of caspases 3 and 7 in NSCLC cells after 24 hours of 2-DG treatment at the indicated concentrations. Bars represent standard error and the studies were repeated twice. FIG. 4B is an immunoblot of cleaved PARP. H23 and H2009 cells were treated for 24 hours with 20 mM of 2-DG. The presence of cleaved PARP is shown in H23, but not in H2009 cells. GAPDH was used as a loading control. FIG. 4C is an immunoblot of LKB1 and KDLKB1 in H23 cells after retroviral infection and puromycin selection. GAPDH was used as a loading control. FIG. 4D is an immunoblot that illustrates 2-DG activation of LKB1 in H23-LKB1, but not H23-KDLKB1 cells. The blot was stripped and probed for AMPK as a loading control. The blot represents two independent studies. FIG. 4E is a graph that illustrates that LKB1, but not KDLKB1 prevents activation of caspase 3 and 7 in H23 cells. H23-LKB1 and H23-KDLKB1 cells were treated for 24 hours with 20 mM 2-DG before analysis as described hereinafter. Bars represent standard error, and the study was repeated twice. FIG. 4F is an immunoblot of cleaved PARP in H23-LKB1 and H23-KDLKB1 after treatment with 20 mM of 2-DG for 24 hours. Blot is representative of two independent studies. GAPDH was used as a loading control.

### DETAILED DESCRIPTION OF THE INVENTION

The present invention relates to the treatment of adenocarcinomas such as NSCLC, colorectal adenoma, prostate and endometrial adenomas wherein the type of treatment provided is largely dependent upon the presence or substantial absence of functionally active LKB1 in the cancer cells. The phrase “functionally active LKB1” is used herein to mean an expressed protein that functions as serine/threonine kinase 11 [STK11; as discussed in Alessi et al., Annual Review of Biochemistry 2006 75:137-163] and having the STK11 enzymatic activity that is usually present in a non-cancerous secretory cell of the same type as that from which the cancerous cell to be treated arose.

Where the adenocarcinoma cells express functionally active LKB1 at usual levels for the cell type, the cancer cells such as NSCLC cells are contacted with both a COX-2-specific inhibitor such as celecoxib, rofecoxib, valdecoxib, parecoxib, lumiracoxib, etoricoxib, and mixtures thereof, and a specific inhibitor of mTOR, such as rapamycin or CCI-779. Those medications need not be co-administered, but are preferably both present in the fluids contacting the adenocarcinoma cells, as occurs upon multiple administrations such that a steady state concentration of both pharmaceuticals is present.

The phrase “COX-2-specific inhibitor” is used herein to differentiate compounds such as celecoxib, rofecoxib, valdecoxib, parecoxib (a prodrug form of valdecoxib), lumiracoxib, etoricoxib (Arcoxia®) from other non-steroidal anti-inflammatory drugs (NSAIDs) such as aspirin, ibuprofen and the like that have substantial activities against both COX-1 and COX-2 enzymes. For example, whereas aspirin is about equipotent at inhibiting COX-2 and COX-1 enzymes in vitro and ibuprofen demonstrates about a sevenfold greater inhibition of COX-2 than of COX-1. On the other hand, COX-2-specific inhibitors such as valdecoxib and rofecoxib are about 300 times more potent at inhibiting COX-2 than COX-1. Celecoxib is approximately 30 times more potent at inhibiting COX-2 than COX-1. The relative degree of selectivity of these compounds for COX-2 over COX-1 is said to be lumiracoxib=etoricoxib>valdecoxib=rofecoxib>>celecoxib [Goodman & Gilson, The Pharmaceutical Basis of Therapeutics, 11 the ed., Brunton et al, eds., McGraw-Hill, Chicago, (2006) page 702]. A contemplated “COX-2-specific inhibitor” is least about 30 times more potent against COX-2 than COX-1. Celecoxib is the preferred “COX-2-specific inhibitor”.

Both types of medicaments are preferably provided at relatively low concentrations; i.e., at a concentration that is less than that normally provided for the FDA-accepted or other accepted use of the medication. For example, celecoxib is typically administered at about 200 to about 400 mg per day. A single oral dose of 200 mg for a human is reported to provide a Cₘₐₓ of 709 ng/ml. [PDR 62 ed., 2008 at page 3064, Table 1.] The concentration contemplated here for a COX-2 inhibitor such as celecoxib is about 25 mg (twice daily) to about 200 mg (once per day), and preferably about 3.8 ng/ml to about 8 μg/ml. Looked at differently, a concentration of about 5 μM to about 50 μM in the contacting fluid is contemplated.

The specific inhibitor of mTOR, a rapamycin-like macrolide such as rapamycin itself Repamune®, Torisel® (CCI-779), or everolimus [RAD-001; or Certican®], is typically present in whole blood at a Cₘₐₓ concentration of 585 ng/ml after a single 25 ml infusion. [PDR 62 ed., 2008 at page 3432.] The concentration contemplated here for a mTOR inhibitor such as Repamune® is about 1 nM to about 100 nM, and preferably about 1 ng/ml to about 100 ng/ml.

Another embodiment contemplates treatment of adenocarcinoma cells such as NSCLC cells that express substantially less than the usual amount of functionally active LKB1. Expression of “substantially less than the usual amount of functionally active LKB1” is an amount that is about 50 percent or less of the amount of functionally active enzyme usually produced by the non-cancerous secretory cells. Here, LKB1 can be not expressed or can be expressed in non-functionally active form due to genetic or epigenetic alterations at the RNA, DNA, or protein level.

An inhibitor of the glycolytic pathway otherwise referred to herein as a cellular metabolism and energetic stress inducer is used for contacting the adenocarcinoma cells in this embodiment. Illustrative such inhibitors include 2-deoxyglucose (2-DG), bromopyruvic acid, 6-aminonicotinamide, oxythiamine chloride, sodium arsenate dibasic heptahydrate, sodium oxamate (oxalic acid monoamide sodium salt), and sodium fluoride, and mixtures of those inhibitors. 2-DG is an exemplary useful medicament for in vivo use and is contemplated for use here in an amount of about 5 to about 50 millimolar (mM), and more preferably at about 2.5 to about 20 mM provided to a host animal. The other glycolytic pathway inhibitors can be used in vivo also, and all can be used in in vitro cellular assays.

Functionally active LKB1 expression can be determined through several methods well-known to those skilled in the art. Illustrative methods include: ELISA, immunohistochemical analysis, epigenetic mapping, DNA sequencing (via traditional Sanger method, pyrosequencing, microarray-based platforms, mass spectrophotometry-based platforms, and/or “next generation” sequencing methods including Solexa, 454, SOLiD, CLiC, and multiplex polony sequencing, as well as “3rd generation” sequencing systems based on single-molecule analysis, etc.), microarray-based gene expression, DNA hybridization, and RNA or DNA PCR-based approaches.

Directing therapy based upon the unique characteristics of each individual's cancer (personalized therapy) has many potential benefits. The recent identification of unique Epidermal Growth Factor Receptor (EGFR) mutations in some NSCLCs has led to the more effective use of EGFR inhibitors [Sharma et al., Nature Reviews 2007 (March) 7(3):169-181]. It is clear that biological approaches to lung cancer have the potential to impact the overall survival from this disease, which historically has lagged behind the success of treatment for cancers of other organ sites. Similarly, understanding molecular characteristics of a cancer has led to attempts at improving the outcomes of traditional chemotherapeutic agents by directing these agents to those cancers most likely to respond [Ceppi et al., Ann Oncol 2006 December 17(12):1818-1825; and Lord et al., Clin Cancer Res 2002 July 8(7):2286-2291].

Currently, the molecular diversity of NSCLC and the complexity of its response to traditional agents suggests that success in improving the outcomes for this disease will rely in part on individualized therapy. To this end, the frequent inactivation of LKB1 in NSCLC has been exploited here to design a potential therapy for patients with these tumors. LKB1 appears to be inactivated/mutated in up to 50 percent of NSCLC [Shah et al., Cancer Research 2008 (May 15) 68(10):3562-3565], representing a large group of patients that could benefit from this therapy. Because LKB1 is associated with the regulation of cellular metabolism, it was thought that a glycolytic inhibitor such as the illustratively used 2-DG could interfere with the survival of cells with inactive LKB1.

2-DG was found to be a potent activator of apoptosis in LKB1 null cells, which confirms previous studies that have demonstrated activation of apoptosis after induction of energetic stress in LKB1 null cells [Corradetti et al., Genes & Development 2004 (Jul. 1) 18(13):1533-1538; Carretero et al., Oncogene 2007 (Mar. 8) 26(11):1616-1625; and Shaw et al., Cancer Cell 2004 July 6(1):91-99]. The 2-DG dependent activation of apoptosis was prevented by re-expression of LKB1 in LKB1 null cells, highlighting the critical role of LKB1 in metabolic adaptation.

During conditions of reduced nutrient availability, cells enter into either autophagy, a process in which energy is derived via catabolism of intracellular organelles, or apoptosis [Lum et al., Nat Rev Mol Cell Biol 2005 6(6):439-448; and Reggiori et al., Current Opinion in Cell Biology 2005 17(4):415-422]. LKB1 activity appears to play a role in this decision, as stabilization of the cell cycle inhibitor, p27/kip1 by LKB1 results in autophagy, instead of apoptosis during energetic stress [Liang et al., Nature Cell Biology 2007 February 9(2):218-224]. Thus, 2-DG treatment may result in autophagy in LKB1 expressing NSCLC cells, an area also currently under investigation.

However, other regulatory functions of LKB1 cannot be discounted as inhibition of mTOR with rapamycin can also prevent cell death during energetic stress in LKB1 null cells [Corradetti et al., Genes & Development 2004 (Jul. 1) 18(13):1533-1538; and Shaw et al., Cancer Cell 2004 July 6(1):91-99], and the inventors have observed increased mTOR activation in LKB1 null cells treated with 2-DG. These findings suggest that the inability to regulate mTOR activity in LKB1 null cells during treatment with 2-DG may result in apoptosis due to undetermined mechanisms. These and other possible mechanisms for 2-DG toxicity in LKB1 null NSCLC cells are being investigated.

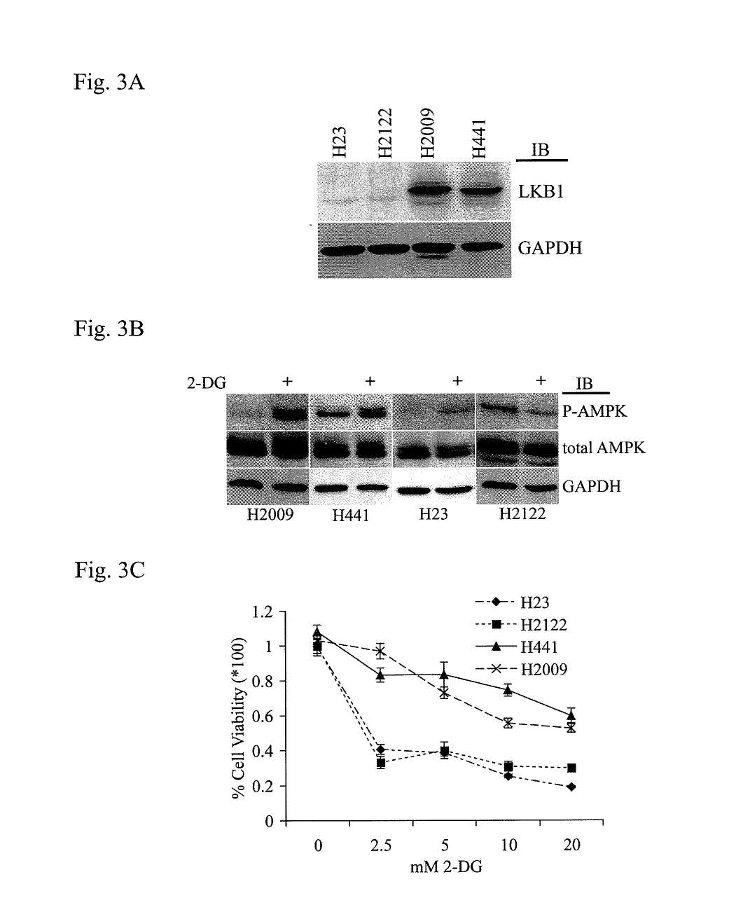

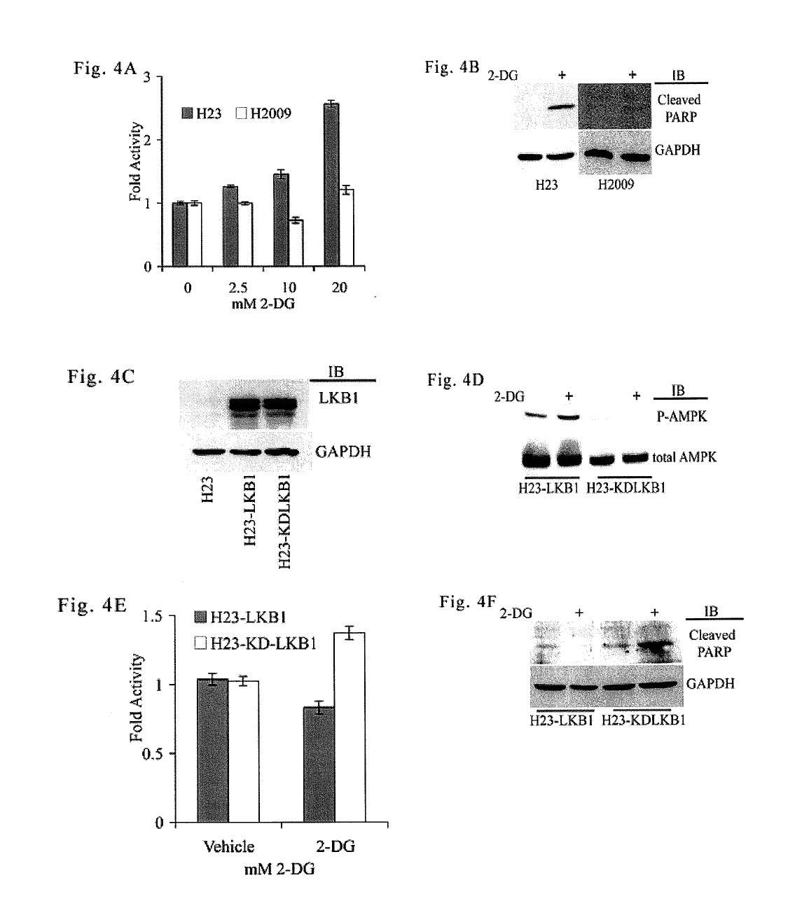

Gene expression analysis revealed dose-dependent changes in BCL-2 and BCL-2 related genes in LKB1 null cells, as well as activation of caspases and caspase function (see FIGS. 3 and 4), suggesting that LKB1 null cells undergo apoptosis via a mitochondrial mediated mechanism. Surprisingly, changes in apoptotic and inflammatory genes in LKB1 expressing cells with 2-DG treatment have also been observed. The increases in these genes suggest that although these cells did not undergo apoptosis in the in vitro system, it is possible that in vivo, the expression of these genes can induce recruitment of inflammatory cells, which can activate apoptosis through receptor mediated pathways. Alternatively, cells in extended periods of autophagy undergo cell death via non-apoptotic type II pathway [Lum et al., Nat Rev Mol Cell Biol 2005 6(6):439-448; and Reggiori et al., Current Opinion in Cell Biology 2005 17(4):415-422], which cannot be determined by the experimental design used here and could generate the observed decreases in cell viability in LKB1 expressing NSCLC after 2-DG treatment (FIG. 3C). Thus, these studies are being extended into in vivo models, as well as studying the effects of glycolytic inhibitors such as 2-DG on LKB1 expressing NSCLC cells.

2-DG is currently undergoing phase I trials in combination with standard chemotherapeutics for the treatment of various solid tumors. The present work indicates that categorizing patients based upon LKB1 expression can yield improved patient response to 2-DG. This treatment paradigm is similar to the use of EGFR inhibitor, Tarceva® (erlotinib), in NSCLC patients with distinct mutations within EGFR, which occur in only 10-15% of NSCLC patients [Sharma et al., Nature Reviews 2007 March 7(3):169-181].

Current statistics have suggested LKB1 loss may occur in 30-50% of NSCLC. However, NSCLC is a heterogeneous disease consisting of large cell carcinoma (LCC), adenocarcinoma, squamous cell carcinoma (SCC) and mixed histology tumors (adenosquamous). Amongst these subtypes, LKB1 loss appears to occur most frequently in adenocarcinoma (34%), with LKB1 loss occurring in SCC (19%), LCC (14%) and adenosquamous (25%) at lower rates [Shah et al., Cancer Research 2008 (May 15) 68(10):3562-3565]. Based upon these findings, studies were focused initially upon adenocarcinoma cell lines.

It is believed that a glycolytic inhibitor such as 2-DG can induce a similar response in SCC, LCC and adenosquamous tumors, a subject currently under investigation by the inventors. Despite the variances of LKB1 loss within the NSCLC subtypes, 2-DG would benefit a substantially larger patient population compared to Tarceva® sensitive NSCLC. Due to the significant lack of treatment options available to NSCLC patients, the targeting of metabolic processes within LKB1 null NSCLC tumor is thought to provide a new avenue for treatment in this disease.

Pharmaceutical Compositions

Although the contemplated medicaments can be administered separately, a pharmaceutical composition for treating adenocarcinoma that contains two medicaments is also contemplated. In one such single composition, a COX-2-specific inhibitor and specific inhibitor of mTOR are dissolved or dispersed in a pharmaceutically acceptable diluent.

A contemplated composition can be used to contact the adenocarcinoma cells in vitro or in vivo. When in vitro contacting is contemplated, the cells are often cells from a biopsy sample that are cultured to determine their LKB1 activity. Such in vitro cultured cells can also be a cell line cultured to assay the effectiveness of a particular composition. For in vivo contacting, a subject to which or whom a contemplated composition is administered can be and preferably is a human, but can also be an ape such as a chimpanzee or gorilla, a laboratory animal such as a monkey, rat, mouse or rabbit, a companion animal such as a dog, cat, horse, or a food animal such as a cow or steer, sheep, lamb, pig, goat, llama or the like.

A contemplated composition is administered to a subject in need of the medication at an LKB1-stimulating amount of COX-2 inhibitor and a mTOR specific inhibiting amount of a rapamycin, or with a growth inhibiting amount of an agent that inhibits cellular metabolism and induces energetic stress (glycolytic inhibitor). Those levels may differ among the several medications contemplated as is well known for each medication.

Illustrative effective dosages for the exemplary medications discussed above can be found in the Physician's Desk Reference, a yearly publication of Thomson Healthcare, such as the 62nd edition published in 2008, as well as in texts such as ALFONSO R. GENNARO. Remington: The Science and Practice of Pharmacy, 20th ed., Lippincott Williams & Wilkins, Baltimore, Md. (2000) (formerly known as Remington's Pharmaceutical Sciences), and Goodman & Gilson's The Pharmacological Basis of Therapeutics, (9th ed.), McGraw-Hill, New York (1996). The amount of a particular medication can also vary depending on the recipient's age and weight as is well-known. Similar concentrations of medicaments can be provided by a liquid suspension for oral administration or a liquid composition for injection, which are also useful in providing a desired plasma or serum concentration.

For preparing pharmaceutical compositions containing useful medicaments, an inert, pharmaceutically acceptable carrier or diluent is used. The diluent can be solid or liquid or gel. Solid form preparations include, for example, powders, tablets, dispersible granules, capsules, cachets, and suppositories.

A solid carrier or diluent can be one or more substances that can also act as a flavoring agent, solubilizer, lubricant, suspending agent, binder, or tablet disintegrating agent; it can also be an encapsulating material.

In powders, the carrier is generally a finely divided solid which is in a mixture with the finely divided active component. In tablets, the active compound is mixed with the carrier having the necessary binding properties in suitable proportions and compacted in the shape and size desired.

For preparing pharmaceutical composition in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and gel or solidify.

Powders and tablets preferably contain between about 5% to about 70% by weight of the active drug ingredient. Suitable diluents (carriers) include, for example, magnesium carbonate, magnesium stearate, talc, lactose, sugar, pectin, dextrin, starch, tragacanth, methyl cellulose, sodium carboxymethyl cellulose, a low-melting wax, cocoa butter and the like.

The pharmaceutical compositions can include the formulation of the active compound with encapsulating material as a carrier providing a capsule in which the active component (with or without other carriers) is surrounded by a carrier, which is thus in association with it. In a similar manner, cachets are also included.

Liquid pharmaceutical compositions include, for example, solutions suitable for oral or parenteral administration, or suspensions, and emulsions suitable for oral administration. Sterile water solutions of the active component or sterile solutions of the active component in solvents comprising water, ethanol, or propylene glycol are examples of liquid compositions suitable for parenteral administration.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Aqueous solutions for oral administration can be prepared by dissolving the active compound in water and adding suitable flavorants, coloring agents, stabilizers, and thickening agents as desired. Aqueous suspensions for oral use can be made by dispersing the finely divided active component in water together with a viscous material such as natural or synthetic gums, resins, methyl cellulose, sodium carboxymethyl cellulose, and other suspending agents known to the pharmaceutical formulation art.

Preferably, the pharmaceutical composition is in unit dosage form. In such form, the composition is divided into unit doses containing appropriate quantities of the active compound. The unit dosage form can be a packaged preparation, the package containing discrete quantities of the preparation, for example; packeted tablets, capsules, and powders in vials or ampules. The unit dosage form can also be a capsule, cachet, or tablet itself, or it can be the appropriate number of any of these packaged forms.

Results

Studies with a COX-2-Specific Inhibitor and a mTOR-Specific Inhibitor

A completed phase I trial of celecoxib with Tarceva® for the treatment of NSCLC has validated this drug combination [Reckamp et al. Clin Cancer Res 2006 12(11 Pt 1):3381-3388]. However, due to the limited patient population responsive to Tarceva® and the prevalence of activating Ras mutations in NSCLC, two NSCLC cell lines with Ras mutations (A549, H2030) were treated with celecoxib and other treating agents. A549 and H2030 cells were treated for 72 hours and percentage of viable cells after treatment was determined.

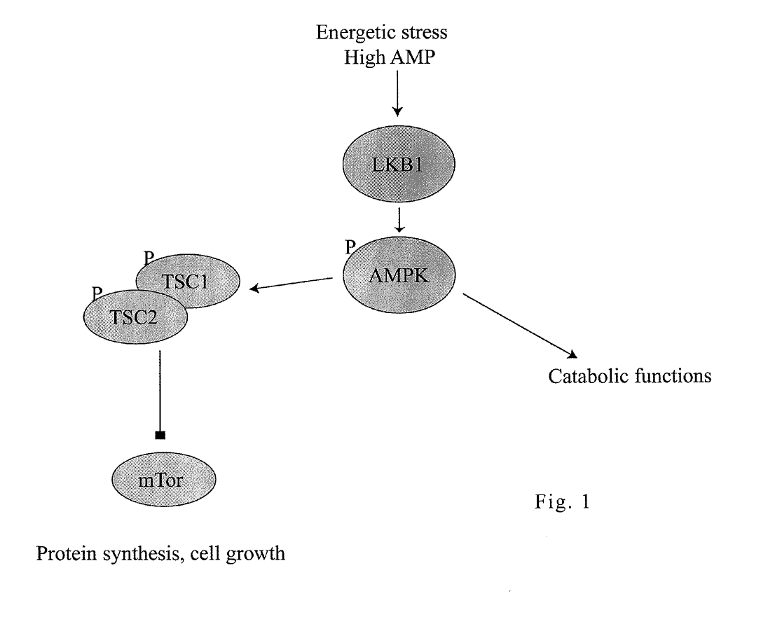

In these screens, it was noticed that although Ras mutant NSCLC cells failed to respond to a combination of celecoxib and Tarceva® as expected, the H2030 cell line responded synergistically to the combination of celecoxib+rapamycin, whereas A549 NSCLC cells did not (FIG. 1). In those studies, celecoxib was present at concentrations of zero, 5, 10, 20 and 40 μM, and was paired with rapamycin at concentrations of zero, 1, 10, 100 and 1000 nM, respectively.

After a search of the literature and the Sanger Institute's Catalog of somatic mutations in cancer (see the Sanger website at sanger.ac.uk/genetics/CGP/cosmic), revealed inactivation of LKB1 in A549 cells, but not in H2030. Further screens in Ras mutant, LKB1+ or LKB1− lung carcinoma cells revealed that LKB1 loss correlated to insensitivity to celecoxib+rapamycin treatment (Table 1). These findings support the hypothesis, and provide a foundation for clinical use of combination targeted therapies.

TABLE 1

| | | Celecoxib + |
|---|---|---|
| | LKB1 | Rapamycin |
| Cell Line | Status | Sensitivity |
| A549 | − | − |
| H460 | − | − |
| A427 | − | − |
| H2030 | + | + |
| Calu−1 | + | + |
| H520 | + | + |

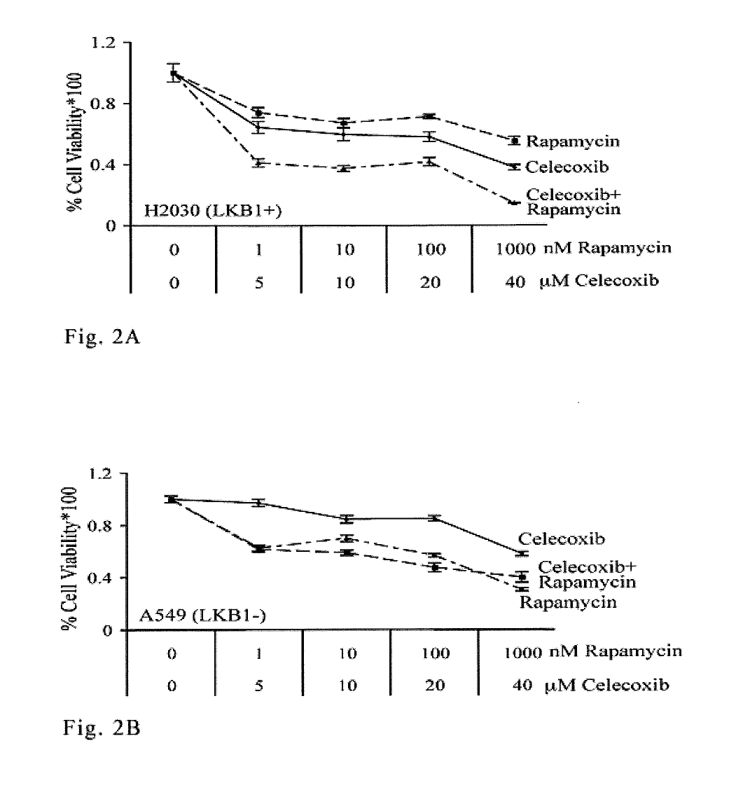

NSCLC cell lines (all Ras mutants) were analyzed as in FIG. 2. LKB1+ cells have a similar cell viability response as H2030 NSCLC cell line.

The Role of LKB1 Expression in Celecoxib+Rapamycin Sensitivity in NSCLC

Activating mutations to the KRAS protein, a common feature in NSCLC [Rodenhuis et al., N Engl J Med 1987 317(15):929-935], results in deregulated cell growth and increases the expression of COX-2 [Dannenberg et al., Cancer Cell 2003 4(6):431-436]. A recent study reported that inactivation of LKB1 functions cooperatively with KRAS mutations in NSCLC [Ji et al., Nature 2007 448(7155):807-810].

However, as somatic loss of LKB1 is not found in the majority of NSCLC cases, decreased LKB1 activity mediated by COX-2 upregulation provides an alternate mechanism for LKB1 deregulation in NSCLC with mutant KRAS. Four NSCLC cell lines with oncogenic KRAS mutations that are either positive or negative for LKB1 (LKB1−: H23, H2122; LKB1+: H441, H2009) are treated with either vehicle, celecoxib, rapamycin or a combination of celecoxib+rapamycin. Cell growth is determined by both cell viability and BrdU incorporation to compare the effects of treatment between LKB1+ and LKB1− NSCLC cell lines. In addition, to confirm the role of LKB1 in celecoxib+rapamycin sensitivity, LKB1 or a kinase-dead LKB1 is reintroduced through viral transfection into the LKB1 null A549 (active KRAS) NSCLC cell line. A549 cells expressing LKB1 (A549-LKB1) or kinase dead-LKB1 (A549-KD-LKB1) are selected with puromycin and the selected cell lines (A549-LKB1, A549-KD-LKB1) are screened as described above.

In addition to decreasing mTOR activity, LKB1 also directs the inhibition of fatty acid synthesis via inhibition of acetyl-CoA-carboxylase (ACC) [Alessi et al., Annu Rev Biochem 2006; 75:137-163]. Inactivation of ACC is due to AMPK dependent phosphorylation, which can be determined by immunoblot with a phosphorylation specific antibody. Likewise, activity of mTOR can be ascertained by immunoblotting levels of phosphorylated S6 kinase, a substrate of mTOR [Guertin et al., Cancer Cell 2007 12 (1):9-22].

To understand what effects celecoxib+rapamycin has upon the downstream targets of LKB1, protein lysates from LKB1+ and LKB1− NSCLC cell lines treated either with vehicle, celecoxib, rapamycin or celecoxib+rapamycin are immunoblotted with phosphorylation specific antibodies to AMPK, ACC and S6 kinase. In addition to investigating these known targets, gene expression profiles of treated A549-LKB1 and A549-KD-LKB1 cell lines are used to investigate and identify any new molecular changes outside the known targets of LKB1 signaling. RNA is isolated from A549-LKB1 and A549-KD-LKB1 cell lines treated with either vehicle, celecoxib, rapamycin or celecoxib+rapamycin and undergoes microarray analysis. Gene expression profiles of each treatment (vehicle, celecoxib, rapamycin, celecoxib+rapamycin) is compared between A549-LKB1 and A549-KD-LKB1 cell lines.

Celecoxib has anti-tumorigenic functions independent of COX-2 inhibition at high doses (>40 mM) [Dannenberg et al., Cancer Cell 2003 4(6):431-436]. One of these is the inhibition of the kinase, phosphoinositide-dependent kinase-1 (PDK-1), which mediates activation of AKT [Kulp et al., Cancer Res 2004 64(4):1444-1451]. As AKT is an activator of mTOR [Guertin et al., Cancer Cell 2007 12 (1):9-22], celecoxib might affect mTOR activation via inhibition of PDK-1. This inhibition combined with the inhibitory activity of LKB1 and rapamycin might result in the sensitivity of LKB1+ cells to celecoxib+rapamycin. To investigate this possibility, expression of COX-2 is reduced by siRNA specific to COX-2 in LKB1+ NSCLC cells. After siRNA treatment, ability of celecoxib+rapamycin treatment to reduce cell growth is evaluated as described above.

Glycolysis Inhibitor Studies

To investigate the role of LKB1 in the effectiveness of 2-DG treatment, four NSCLC cell lines (H23, H2122, H441, H2009) were screened by immunoblot for LKB1 expression (FIG. 3A). Exposure of cells to 20 mM of 2-DG induced LKB1 dependent activation of AMPK, confirming published data (FIG. 3B).

Gene expression profiles of 2-DG-treated cells revealed an increase in the expression of pro-apoptotic markers in LKB1 negative cell lines, whereas LKB1 positive lines demonstrated no changes in expression. 2-DG therapy thus can be a useful agent in the treatment of patients with NSCLC. Loss of LKB1 is associated with a marked increase in susceptibility to 2-DG treatment in NSCLC lines, even at low doses. Determination of LKB1 status can help direct therapy to those patients most likely to benefit from this novel approach.

Because LKB1-AMPK signaling can affect cell growth or cell number, the number of viable LKB1 null and LKB1 positive cells were compared after treatment with 2-DG for a range of concentrations, over time. After 48 hours of treatment, both LKB1 positive and LKB1 negative cells showed a significant decrease in cell viability at high doses (20 mM) of 2-DG (FIG. 3C), thus confirming 2-DG's ability to limit cell number. However, LKB1 null cells showed a significant decrease in cell viability for all doses tested, even at the lowest dose (2.5 mM) compared to LKB1 positive cells (p=5.73×10⁻¹³). 2-DG treatment induced apoptosis in LKB1 negative cell lines, but not in LKB1 positive cells (FIG. 3C). These findings suggest that LKB1 null NSCLC cells are significantly more sensitive to treatment with 2-DG than LKB1 positive cells and that 2-DG might induce a different response in LKB1 null NSCLC cells.

Activation of energetic stress by glucose starvation results in the induction of apoptosis or programmed cell death in both LKB1 null NSCLC cells and LKB1 null mouse embryonic fibroblasts [Corradetti et al., Genes & Development 2004 (Jul. 1) 18(13):1533-1538; Carretero et al., Oncogene 2007 (Mar. 8) 26(11):1616-1625; and Shaw et al., Cancer Cell 2004 July 6(1):91-99]. Because 2-DG had a more significant effect upon cell viability in LKB1 null, but not LKB1 positive NSCLC cell lines, it was hypothesized that 2-DG might alternately induce apoptotic pathways in LKB1 null cell lines, reducing cell number.

To test this hypothesis, apoptosis-related transcripts of H23 (LKB1 null) and H2009 (LKB1 positive) cells were compared by treatment with either low (2.5 mM) or high dose (20 mM) 2-DG or vehicle (PBS) for 6 hours followed by microarray-based assessment of gene expression. Expression analysis revealed dramatic changes in a variety of genes due to 2-DG treatments in both H2009 and H23. Interestingly, although little difference in expression of the pro-apoptotic protein, Fas ligand and its related proteins was found, dose dependent changes in the expression of the anti-apoptotic protein, BCL-2 and several BCL-2 interacting proteins were noted in LKB1 null H23 cells.

Taken together, these results suggested an activation of apoptosis within H23 LKB1 null NSCLC cells, but not H2009 (LKB1 positive) cells, by 2-DG. To confirm that 2-DG activated apoptosis in LKB1 null, but not LKB1 positive NSCLC cells, H23 and H2009 were treated with increasing doses of 2-DG for 24 hours and assayed for activation of caspases 3 and 7. Treatment with 2-DG induced a dose dependent activation of caspases 3 and 7 in H23 cells, consistent with the gene expression data (FIG. 4A). Further, immunoblot analysis of cleaved PARP, a marker of apoptosis, was present in 2-DG treated H23 cells, but not in H2009 cells (FIG. 4B). Thus, these findings demonstrate that 2-DG induces apoptosis in LKB1 null, but not in LKB1 positive cells.

To understand if the lack of LKB1 function was responsible for the effects of 2-DG on NSCLC cells, a FLAG tagged LKB1 was re-introduced into H23 cells via retroviral infection. In addition, an inactive, kinase dead form of LKB1 (KDLKB1) was also introduced using the same retroviral vector as a negative control. H23 cells were infected with either FLAG tagged LKB1 or KDLKB1 retroviruses and underwent puromycin selection. The resulting cell lines, H23-LKB1 and H23-KDLKB1, both expressed LKB1, as shown by western blot (FIG. 4C). However, only H23-LKB1 is capable of phosphorylating AMPK when treated with 2-DG, whereas H23-KDLKB1 does not (FIG. 4D). Re-expression of LKB1 in H23 cells prevented 2-DG induced activation of caspase 3 and 7, but not in H23 cells expressing KDLKB1 (FIG. 4E). In addition, re-expression of LKB1 in H23 cells also prevented cleavage of PARP after treatment with 2-DG, but not in H23-KDLKB1 cells (FIG. 4F). In sum, these findings demonstrate that LKB1 function is critical in determining the effects of 2-DG in NSCLC cells.

Methods

Studies with a COX-2-Specific Inhibitor and a mTOR-Specific Inhibitor

A. Cell Viability and Growth:

NSCLC cells are plated onto 96 well plates and allowed to attach. Celecoxib and rapamycin are diluted at varying amounts in low glucose media (>10 mM) to replicate physiological levels of glucose within the tumor microenvironment and added to cells. After 72 hours, cell viability is assessed using the CellTiter™ blue kit (Promega) and read on a DTX880 plate reader (Beckman-Coulter). For cell growth, BrdU is added at 48 hours after drug treatment and pulsed for 24 hours before determination of BrdU incorporation according to manufacturer's instructions (Millipore).

B. Immunoblot of LKB1 Downstream Targets:

NSCLC cell lines are treated in low glucose media as described above. Protein lysates from predetermined time points are separated by SDS-PAGE and transferred to nitrocellulose for immunoblotting with specific antibodies to phosphorylated AMPK, ACC and S6 kinase (Cell Signaling Technologies). After development with ECL plus (GE Healthcare), blots are stripped and probed with antibodies against AMPK, ACC and S6 kinase for loading controls.

C. Microarray Analysis:

RNA is isolated from A549-LKB1 and A549-KD-LKB1 cell lines with Trizol reagent (Invitrogen) after treatment as described for immunoblotting experiments. 500 ng of RNA is used for each microarray labeling reaction using the Agilent Low RNA Input Linear Amplification Kit PLUS to generate CY3 labeled probes and purified using a modified Qiagen RNeasy Mini Kit protocol. Probes are hybridized to Agilent 4×44K Multiplex Whole Human Genome One-Color Oligo Microarrays according to manufacturer specifications. Slides are scanned using the Agilent Microarray Scanner (model G2505B) and processed with Agilent's Feature Extraction software (v. 9.5.1). All arrays are quality controlled for a minimum median hybridization intensity of greater than 85 units and a maximum average background level of 50 units in each channel (scale 0-65,000 units).

Celecoxib+Rapamycin Combinational Therapy on LKB1 Expressing NSCLC Cells in Vivo

Preliminary results indicate that treatment of LKB1-expressing NSCLC cells with celecoxib and rapamycin has a negative effect upon cell growth in vitro, suggesting that this combination of drugs can be effective in decreasing growth in vivo. Severe combined immuno-deficient (SCID) mice, a commonly used model in cancer biology, are used to test this hypothesis. After injection into SCID mice, human cancer cells readily form xenograft tumors and are used to monitor a variety of characteristics of cancer (growth, angiogenesis, apoptosis, signaling).

To assess the effects of celecoxib and rapamycin in combination on the in vivo growth of LKB1 expressing NSCLC cells, A549-LKB1 and A549-KD-LKB1 cells are injected into the hind limb and permitted to form a palpable tumor. Tumor-bearing mice are randomized into four groups: vehicle, celecoxib, rapamycin, celecoxib+rapamycin, for daily treatment. Growth of tumors is measured daily for four weeks, at which point animals are sacrificed and tumors harvested and separated equally for analysis by immunoblot, microarray and immunohistochemistry as described above.

Because the mouse hind limb does not represent the normal environment of the lung, celecoxib+rapamycin treatment can show limited efficacy. To address this question, the efficacy of celecoxib+rapamycin treatment is assessed using a lung orthotopic mode [Sievers et al., J Thorac Cardiovasc Surg 2005 129(6):1242-1249]. A549-LKB1 and A549-KD-LKB1 cells are injected into the left lung lobe of SCID mice. After 3 weeks, mice are randomized into treatment groups (vehicle, celecoxib, rapamycin, celecoxib+rapamycin) and treated. After 4 weeks, mice are sacrificed and lungs weighed to determine the gross tumor weight. Tumors are divided for analysis as described above.

A. Xenograft Model:

One million cells (A549-LKB1 or A549-KD-LKB1) are injected subcutaneously into the hind limb of a 6-week-old SCID mouse. Mice are checked three times a week for palpable tumor. Treatment begins once a palpable tumor has formed. Mice are treated once daily by oral gavage with celecoxib (100 mg/kg), rapamycin (1 mg/kg) or a combination of both. Tumors are measured with calipers and tumor volume is determined by calculating with the formula, (4/3π H×L×W). After four weeks, or until tumor reaches 2 cm³, animals are sacrificed. Tumors are divided equally for immunoblotting, microarray analysis or 4% neutral buffered formalin for paraffin blocks. Samples are analyzed as described above.

Treatments with Inhibitors of Cellular Metabolism and Energetic Stress Inducers

2-Deoxyglucose (2-DG) preferentially targets tumor cells due to their increased glucose uptake. Mechanistically, 2-DG inhibits cellular metabolism and induces energetic stress, resulting in decreased cellular viability. The serine-threonine kinase, LKB1 regulates cellular metabolism and can play an important role in 2-DG induced cellular damage. Energetic stress activates LKB1 and induces cell cycle arrest as a means to conserve energy. Conversely, cells that lack LKB1 fail to react to such stress and undergo cell death.

Somatic loss of LKB1 has been found to occur in about 30 percent of NSCLC, indicating an increased cellular susceptibility to 2-DG therapy in LKB1 null cells and in patients whose cancers have such cells. It was believed that the absence of LKB1 from adenocarcinoma cells would increase the susceptibility of those cells to treatment with 2-DG.

Glycolysis Inhibitor Studies

LKB1 negative (H23, H2122) and positive (H2009, H441) NSCLC cell lines were treated with decreasing doses of 2-deoxyglucose (2-DG) for 72 hours. Cell viability, markers of apoptosis (e.g. PARP, Caspases), and gene expression profiles were evaluated from treated versus untreated samples.

Reagents and Cell Culture

H23, H2009, H2122, and H441 lung adenocarcinoma cell lines were obtained from ATCC and maintained in RPMI 1640 (Invitrogen)/10% Fetal Bovine Serum under standard tissue culture conditions. Antibodies to LKB1, phosphorylated AMPK, AMPK, and cleaved PARP were purchased from Cell Signaling Technologies (MA). CellTiter Blue® and Caspase-Glo® 3/7 kits were purchased from Promega (WI). 2-Deoxyglucose (2-DG) was purchased from Sigma (MO) and diluted to a 1M stock in sterile phosphate buffered saline (PBS). The pBabe retroviral constructs containing FLAG tagged full length LKB1 and the kinase dead LKB1 originated in Dr. Lewis Cantley's laboratory (Harvard Medical School, MA) and were obtained from Addgene.org (MA).

Cell Lysis and Immunoblotting

Cells were incubated on ice for 30 minutes with a lysis buffer containing 10 mM Tris-HCL, 150 mM NaCl and 1% IGEPAL (Sigma). Immediately before use, a protease inhibitor cocktail (Sigma) and phosphatase cocktails I and II (Sigma) were added to the lysis buffer. Lysates were transferred to tubes and insoluble material was pelleted by centrifugation. Protein concentration was determined using the Bio-Rad DC protein kit. For immunoblotting, 100 μg of total protein lysate was separated by SDS-PAGE and transferred to nitrocellulose. Blots were blocked in 5% milk/Tris buffered saline/0.1% Tween 20 (TBST) and primary antibodies were diluted in 5% Bovine Serum Albumin (Sigma)/TBST and incubated overnight (about 18 hours) at 4° C. Blots were developed with ECL plus (GE) and visualized on a Kodak Image station.

Cell Viability

Cell lines were plated at a density of 2500 cells per well in 96 well plates. After attachment, 2-DG was diluted in media at indicated concentrations and added to wells. Treatment was carried out for 48 hours in standard cell culture conditions, before cell viability was assessed using the CellTiter® Blue kit (Promega) according to manufacturer's instructions and read on a Beckman Coulter DTX 880. The percentage of viable cells was determined by normalizing treated samples to vehicle (PBS).

Caspase 3/7 Activity

Cell lines were plated at a density of 5000 cells per well in 96 well plates. After attachment, cells were treated with either 2-DG or vehicle for 24 hours. Activity of caspases 3 and 7 were determined using a Caspase-Glo® 3/7 kit (Promega) according to manufacturer's instructions and scanned using a Beckman Coulter DTX 880 plate reader. Relative light units from treated samples were normalized to vehicle controls to generate fold activity of caspases 3 and 7.

Gene Expression Profiling

Total RNA was extracted from cells using TRIzol (Invitrogen) followed by RNeasy Mini Kit purification (Qiagen) according to manufacturers specifications. Spectrophotometric analysis was performed on a NanoDrop ND-1000 (Thermo Scientific) and RNA quality was assessed on a BioAnalyzer 2100 (Agilent) using strict RNA integrity QC cutoffs. 500 ng of RNA was labeled and hybridized (in duplicate) to Agilent 4×44K Whole Human Genome One-Color Oligo Microarrays according to manufacturer specifications. Slides were scanned using the Agilent Microarray Scanner (model G2505B) and processed with Agilent's Feature Extraction software (v. 9.5.1). Comparative and statistical analyses for gene expression profiles, were carried out using GeneSpring 7.3. Apoptosis-related genes of interest were identified by key word search (“apoptosis”) followed by unsupervised hierarchical clustering. Significance values were determined by comparing expression of all LKB1+ samples (grouped) to all LKB1− samples (grouped) for each gene using the Student's t-test and a p<0.05 cutoff after multiple testing correction using the false discovery rate (FDR) method.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method of treating adenocarcinoma cells that express functionally active LKB1 that comprises contacting said cells with a LKB1-stimulating amount of a COX-2-specific inhibitor in combination with an inhibiting amount of a specific inhibitor of mTOR.

2. The method according to claim 1, wherein said adenocarcinoma cells are non-small cell lung cancer.

3. The method according to claim 1, wherein said COX-2-specific inhibitor is selected from the group consisting of celecoxib, rofecoxib, valdecoxib, parecoxib, lumiracoxib, etoricoxib and mixtures thereof.

4. The method according to claim 1, wherein said specific inhibitor of mTOR is a rapamycin-like macrolide.

5. The method according to claim 1, wherein said contact is carried out in vitro.

6. The method according to claim 1, wherein said contact is carried out in vivo.

7. The method according to claim 6, wherein said in vivo contact is carried out multiple times over a period of months or years.

8. A method of treating adenocarcinoma cells that express about 50 percent or less of the normal, functional LKB1 expressed by non-transformed cells of the same type that comprises contacting those cells with growth inhibiting amount of an agent that inhibits cellular metabolism and induces energetic stress.

9. The method according to claim 8, wherein said adenocarcinoma cells are non-small cell lung cancer.

10. The method according to claim 8, wherein said agent that inhibits cellular metabolism and induces energetic stress is 2-deoxyglucose.

11. The method according to claim 8, wherein said contact is carried out in vitro.

12. The method according to claim 8, wherein said contact is carried out in vivo.

13. The method according to claim 12, wherein said in vivo contact is carried out multiple times over a period of months or years.

14. The method according to claim 8, wherein said agent that inhibits cellular metabolism and induces energetic stress is selected from the group consisting of 2-deoxyglucose, bromopyruvic acid, 6-aminonicotinamide, oxythiamine chloride, sodium arsenate dibasic heptahydrate, sodium oxamate, sodium fluoride, and mixtures thereof.

15. A method of treating adenocarcinoma cells that comprises contacting the cells with one or the other of a pharmaceutical composition containing (1) a LKB1-stimulating amount of a COX-2-specific inhibitor in combination with an inhibiting amount of a specific inhibitor of mTOR, or (2) a growth inhibiting amount of an agent that inhibits cellular metabolism and induces energetic stress, wherein said cells that express functionally active LKB1 are contacted with (1), and cells that express about 25 percent or less of the normal, functional LKB1 expressed by non-transformed cells of the same type are contacted with (2).

16. The method according to claim 15, wherein said contact is carried out in vitro.

17. The method according to claim 15, wherein said contact is carried out in vivo.

18. The method according to claim 17, wherein said in vivo contact is carried out multiple times over a period of months or years.

19. The method according to claim 15, wherein said COX-2-specific inhibitor is celecoxib, rofecoxib or valdecoxib, and said specific inhibitor of mTOR is a rapamycin-like macrolide.

20. The method according to claim 15, wherein said agent that inhibits cellular metabolism and induces energetic stress is selected from the group consisting of 2-deoxyglucose, bromopyruvic acid, 6-aminonicotinamide, oxythiamine chloride, sodium arsenate dibasic heptahydrate, sodium oxamate, sodium fluoride, and mixtures thereof.
